(19) United States
(12) Patent Application Publication
(10) Pub. No.: US 2026/0068922 A1
(43) Pub. Date: Mar. 12, 2026

(54) LIQUID COMPOSITION FOR AN ELECTRONIC VAPOR DEVICE

(71) Applicant: Cronos Group Inc., Toronto, CA (US)

(72) Inventor: Gordon Hagen, Toronto (CA)

(21) Appl. No.: 19/324,429

(22) Filed: Sep. 10, 2025

Related U.S. Application Data
(62) Division of application No. 17/292,686, now abandoned.
(60) Provisional application No. 62/758,218, filed on Nov. 9, 2018.

(51) Int. Cl. A24B 15/167; A24B 15/30; A24F 40/10; A61K 36/185

(52) U.S. Cl. CPC A24B 15/167; A24B 15/303; A24F 40/10; A61K 36/3482

(57) ABSTRACT

A liquid composition for an electronic vaporization device, consisting essentially of an active inhalable source and a terpene source, is disclosed. A liquid composition for an electronic vaporization device is provided consisting essentially of greater than about 60 wt % cannabinoids, from about 5 to about 15 wt % terpenes and less than about 35 wt % non-cannabinoid, non-terpene cannabis phytochemicals. A method is provided for obtaining such a composition. An electronic vaping device and a cartridge for an electronic vaping device containing the composition are also disclosed.

### FIELD

The present disclosure relates to a liquid composition for an electronic vaping device. In particular, the present disclosure relates to a liquid composition comprising phytocannabinoids and/or terpenes.

### INTRODUCTION

Cannabis is a genus of flowering plants that has been used by humans for various purposes, such as medicines, ritual, recreation and textiles. The flowers of the cannabis plant include glandular trichomes, in which phytocannabinoids are produced.

Over 100 phytocannabinoids produced by the cannabis plant have been identified to date. Two notable phytocannabinoids are Δ⁹-tetrahydrocannabinolic acid (THCA) and cannabidiolic acid (CBDA). THCA, when decarboxylated, is transformed into Δ⁹-tetrahydrocannabinol (THC). THC is a psychoactive substance that users may use in order to get a “high” when the cannabis flower is smoked, but has also been shown to be useful for other purposes, such as an appetite stimulant for people with AIDS and an antiemetic for people undergoing chemotherapy (based, at least, on product monographs for dronabinol approved by the FDA). CBDA, when decarboxylated, is transformed into cannabidiol (CBD). CBD has been shown to be useful for treating certain types of epilepsy (based, at least, on product monographs for cannabidiol approved by the FDA), and has other purported effects. Other cannabinoids have also been purported to have physiological, neurological, and/or therapeutic effects.

In addition to phytocannabinoids, the cannabis plant also produces terpenes and terpenoids (collectively “terpenes” unless context dictates otherwise). Terpenes are organic compounds produced in a variety of plants, many of which are consumed in human diets and/or used in perfumes. They contribute to the aromas and flavors of different cannabis cultivars. The terpenes found in cannabis share a precursor with phytocannabinoids. These terpenes can include caryophyllene (also found in black pepper, cloves, and oregano); pinene (also found in pine needles, rosemary, and basil); limonene (also found in citrus peels); myrcene (also found in hops, lemongrass, and mangoes); linalool (also found in lavender, coriander, and cinnamon); and terpinolene (also found in allspice, conifers, and sage).

Cannabis varieties are often differentiated based on their phytocannabinoid and terpene profiles. It has been postulated that combinations of cannabinoids and terpenes found in cannabis contribute to the “entourage effect”, where the binding of at least one cannabinoid to a cannabinoid receptor is modulated by the combinations of cannabinoids and terpenes, such as by moderating the psychoactive effects of THC (see, for example, Ethan B Russo, “Taming THC: potential cannabis synergy and phytocannabinoid-terpenoid entourage effects”, Br J Pharmacol. 2011 August 163 (7): 1344-1364). For example, users of dronabinol, a synthetic version of THC, have reported that dronabinol is less effective in treating certain symptom than using cannabis. It has been postulated that at least part of the reason for this decreased effectiveness is due to the absence of terpenes in dronabinol.

Currently, the most common method of utilizing cannabis is through inhalation of combusted dried cannabis flower. However, reactions occurring during the combustion of the dried flower can result in the formation of undesirable by-products. By-products can include formaldehyde, acetaldehyde, acrolein and other potentially carcinogenic compounds.

In other combusted plant products, such as tobacco products, some users have switched from traditional combustion cigarettes to alternative delivery mechanisms in an effort to reduce their exposure to such compounds. For example, an alternative to tobacco cigarettes is an electronic vaping device (a “vape” or an “e-cigarette”). In tobacco, the active inhalable ingredient (“AII”) is nicotine. Electronic vaping devices vaporize a liquid composition containing AIIs (such as nicotine) into a “vapor” in order to permit inhalation by the user.

For example, a vape (FIG. 1) can include several elements, including a vaporizing element, such as a heater 102 powered by power source 112, and a reservoir 104 for holding the liquid composition. The liquid composition is transported from the reservoir 104 to the vaporizing element 102 via a liquid composition transport 108, which induces vaporization of the liquid formulation, thereby producing a vapor. A user can inhale the vapor, by taking the vapor through a channel 110, and any AIIs contained therein, into the user's body. These vapors are often produced at temperatures such that the formation of potentially harmful by-products is reduced as compared to a conventionally combusted analog. Apertures 106 allow air to flow through channel 110 and act as a carrier for the vapor.

However, it has been found that conventional liquid compositions containing cannabinoids may be perceived as “harsh” and/or have unpleasant flavors. In addition, conventional liquid compositions may contain carriers that may not be desirable for inhalation.

There is a need for improved liquid compositions for use in electronic vaping devices.

### SUMMARY

Aspects of the present disclosure relate to liquid formulations for electronic vaporization devices.

In accordance with one aspect, there is provided a liquid composition for an electronic vaporization device consisting essentially of an active inhalable source and a terpene material.

In accordance with another aspect, there is provided a liquid composition for an electronic vaporization device consisting essentially of greater than about 60 wt % cannabinoids; from about 5 to about 15 wt % terpenes; and less than about 35 wt % non-cannabinoid, non-terpene cannabis phytochemicals.

In accordance with another aspect, there is provided a process of obtaining a composition from starting materials, wherein the composition comprises at least 65 weight % cannabinoid material and at least 5 weight % terpene material; the cannabinoid material consists of at least one cannabinoid; the terpene material consists of at least one terpene; and the converting is effected at a temperature of less than 160° C.

In accordance with another aspect, there is provided an electronic vaping device including a liquid composition as described herein.

In accordance with another aspect, there is provided a cartridge for an electronic vaping device including a liquid composition as described herein.

### DESCRIPTION OF DRAWINGS

In the drawings, embodiments are illustrated by way of example. It is to be expressly understood that the description and figures are only for the purpose of illustration and as an aid to understanding and that the invention should not be limited to the illustrative embodiments provided herein.

FIG. 1 is a schematic diagram of an electronic vapor device to which a liquid composition according to the present disclosure can be loaded.

FIG. 2 is a schematic diagram showing an experimental set up for analyzing cannabinoid and carbonyl generation of vape compositions.

FIG. 3 is a graph showing the amounts of cannabinoids generated using the experimental set up shown in FIG. 2 and described in Example 3.

FIG. 4 is a graph showing the amounts of formaldehyde generated using the experimental set up shown in FIG. 2 and described in Example 3.

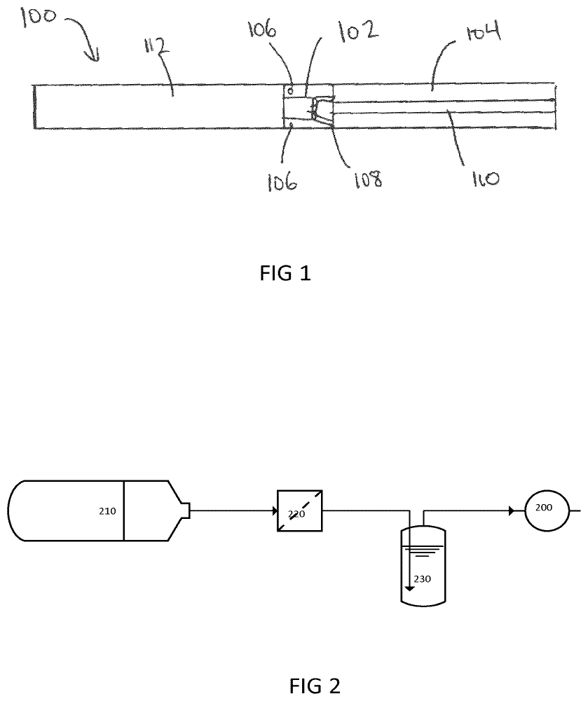

### DETAILED DESCRIPTION

The present inventions now will be described more fully with reference to the drawings, in which some, but not all embodiments of the inventions are shown. The description may be embodied in many different forms and should not be construed as being limited to the embodiments set forth herein. Like numbers refer to like elements throughout.

### Definitions

Unless defined otherwise, all technical and scientific terms used herein have the same meaning as commonly understood by one of ordinary skill in the art to which the invention pertains. Although any methods and materials similar or equivalent to those described herein can be used in the practice for testing of the present invention, specific examples of appropriate materials and methods are described herein.

As used herein, the singular forms “a”, “an”, and “the” refer to both the singular as well as plural, unless the context clearly indicates otherwise. For example, the term “a flower” includes single or plural flowers and can be considered equivalent to the phrase “at least one flower”.

The dimensions and values disclosed herein should not to be understood as being strictly limited to the exact numerical values recited. Rather, unless otherwise specified, each dimension or value is intended to mean both the recited value and a functionally equivalent range surrounding that value. For example, a dimension disclosed as “40 mm” is intended to mean “about 40 mm”.

As used herein, the word “about” means, when used in connection with a numerical value, that the associated numerical value includes a tolerance of +10% around the stated numerical value. Moreover, when reference is made to percentages in this specification, it is intended that those percentages are based on weight, i.e., weight percentages, unless otherwise indicated. The expression “up to” includes amounts of zero to the expressed upper limit and all values therebetween. When ranges are specified, the range includes all values therebetween, such as increments of 0.1%.

As used herein, the term “material”, as it relates to chemical compounds, refers to a composition that consists of a particularly named compounds or class of compounds, and includes both pure substances and mixtures of different compounds. For example, a “cannabinoid material” consists of one or more distinct cannabinoid molecules. Similarly, a “terpene material” consists of one or more distinct terpene molecules.

As used herein, the term “source”, as it relates to chemical compounds, refers to a composition that includes one or more distinct compounds, and includes both pure substances and mixtures of different compounds. Accordingly, a “cannabinoid source” comprises of one or more distinct cannabinoid molecules. Similarly, a “terpene source” comprises one or more distinct terpene molecules.

As used herein, the term “natural cannabinoid source” means a cannabinoid source derived from cannabis, and can include a cannabis extract, a cannabis distillate, a cannabis isolate. In addition to cannabinoids, a natural cannabinoid source can include other phytochemicals produced in cannabis, such as sugars, fats, waxes and chlorophyll, and residual processing chemicals, such as solvents.

As used herein, “cannabis extract” means a product obtained through leaching or extraction from cannabis. Extraction processes generally involve the use of a solvent to dissolve a desired substance. Where cannabinoids are the desired substance, solvents that can be employed include aliphatic hydrocarbons (such as propane, butane), alcohols (such as ethanol), petroleum ether, naphtha, olive oil, carbon dioxide (including supercritical and subcritical CO₂), chloroform, or combinations thereof. See for example, Luigi L Romano and Arno Hazekamp, “Cannabis Oil: chemical evaluation of an upcoming cannabis-based medicine” (2013) 1:1 Cannabinoids 1; H. Perrotin-Brunel et al, “Supercritical Fluid Extraction of Cannabis: Experiments and Modeling of the Process Design” 2010 ISASF-Graz 1-6; Carla Da Porto et al “Separation of aroma compounds from industrial hemp inflorescences (Cannabis sativa L.) by supercritical CO₂ extraction and on-line fractionation”, (2014) 58 Ind Crop Prod. 99; Laura J. Rovetto and Niccolo V. Aieta, “Supercritical carbon dioxide extraction of cannabinoids from Cannabis sativa L.”, (2017) 129 J Supercrit Fluid. 16; Michelle Sexton et al “Evaluation of Cannabinoid and Terpenoid Content: Cannabis Flower Compared to Supercritical CO₂ Concentrate” (2018) 84:4 Planta Med. 234. A cannabis cannabinoid extract includes less than about 70%, 75%, 80%, or 85% of phytocannabinoids, with the balance being other cannabis phytochemicals, such as terpenes, fats, waxes, sugars, chlorophyll, and residual extraction solvent. A cannabis terpene extract includes at least about 70%, 75%, 80%, 85%, 90%, or 95% of terpenes, with the balance being other cannabis phytochemicals, such as terpenes, fats, waxes, sugars, chlorophyll, and residual extraction solvent.

Cannabis extracts are optionally winterized. In winterization, cannabis extract is admixed with a solvent, typically ethanol, and cooled. The cooling causes certain phytochemicals, preferably fats, waxes, to precipitate, allowing them to be filtered from the admixture. The filtered admixture can then undergo a solvent removal, such as through evaporation, to obtain a winterized extract. Cannabis extracts can be commercially obtained, for example, from MediPharm Labs Corp, Valens GroWorks Corp, Neptune Wellness Solutions Inc., or Heritage Cannabis Holdings Corp.

As used herein “cannabis distillate” means a product obtained through the distillation of cannabis or a preparation thereof (typically, a cannabis extract). Distillation of cannabis is typically used to concentrate cannabinoids. A distillation input is often heated to a temperature of at least 140° C., 150° C., 160° C., 170° C., 180° C., 190° C., 200° C., 250° C., 300° C. or 350° C. A cannabis cannabinoid distillate includes greater than 85%, 86%, 87%, 88%, 89%, 90%, 91%, 92%, 93%, or 94% cannabinoids, but less than 95% cannabinoids. A cannabis cannabinoid distillate includes at least about 15%, 14%, 13%, 12%, 11%, 10%, 9%, 8%, 7%, 6% or 5% non-cannabinoid cannabis phytochemicals. Due to similarities in properties of phytocannabinoids, distillation is generally not able to concentrate an individual cannabinoid.

As used herein, the term “cannabinoid isolate” means a product obtained through a process to purify a selected phytocannabinoid from the cannabis plant such that the product contains greater than 95%, 96%, 97%, 98%, 99%, or 99.5% of the selected phytocannabinoid. Cannabis cannabinoid isolates can be obtained, for example, by using chromatographic or precipitation techniques. A cannabis cannabinoid isolate includes up to 5%, 4%, 3%, 2% or 1% of impurities. Such impurities can include non-desired phytocannabinoids, other non-cannabinoid cannabis phytochemicals or trace solvents.

As used herein, the term “synthetic” before a compound or class of compounds mean that the compound or class of compounds is derived from chemical synthesis and not in vivo or in planta, and have a purity of greater than 95%. Synthetic phytocannabinoids can be prepared according to methods known in the art. See for example, GR Handrick et al, “Hashish: Synthesis of (−)-Δ⁹-tetrahydrocannabinol (THC) and its biologically potent metabolite 3′-hydroxy-Δ⁹-THC”, (1979) 20:8 Tetrahedron Letters 681; Raphael Mechoulam et al, “Carboxylation of resorcinols with methyl magnesium carbonate. Synthesis of cannabinoid acids” (1969) 7 J Chem Soc D 343. Synthetic phytocannabinoids includes semi-synthetic cannabinoids wherein a cannabinoid or a precursor thereof is obtained from the cannabis plant. For example, CBD from cannabis can be converted to THC through acid catalysis; and cannabigerolic acid from cannabis can be converted to THCA, CBDA or CBCA using cannabis oxidoreductases secreted from genetically modified Pichia pastoris (see, for example, Futoshi Taura, “Production of Δ¹-tetrahydrocannabinolic acid by the biosynthetic enzyme secreted from transgenic Pichia pastoris” (2007) 361 Biochem and Biophys Res Comm 675; and U.S. Pat. No. 9,394,510 to Winnicki et al).

As used herein, the term “biosynthetic” before a compound or class of compounds mean that the compound or class of compounds is derived from a living organism that does not natively produce the compound or class of compounds, and have a purity of greater than 95%, 96%, 97%, 98%, 99%, or 99.5%. For example, a yeast or bacteria can be engineered to produce phytocannabinoids by insertion of the cannabinoid biosynthesis pathway. Similarly, a yeast or bacteria can be engineered to produce terpenes by upregulation of one or more steps in the mevalonate pathway and insertion of particular terpene synthases.

As used herein, the term “cannabis” means a plant of genus Cannabis. Unless the context clearly indicates otherwise, includes any part of the plant, such as the stalks, branches, leaves, flowers and seed. Cannabis is an annual, dioecious, flowering herb. Cannabis flowers contain trichomes, which are structures where certain compounds, including phytocannabinoids and terpenes, are secreted. Various taxonomical structures of plants of genus Cannabis have been proposed, such as those including a single species, Cannabis sativa, or as multiple species that additionally includes Cannabis indica and/or Cannabis ruderalis, which are considered subspecies under the single species classification.

As used herein, the term “cannabinoid” means any molecule that can bind to or modulate the activity of an endocannabinoid receptor (e.g. a CB1 receptor, a CB2 receptor, or both). Ligands for endocannabinoid receptors include phytocannabinoids, synthetic cannabinoids, and endocannabinoids.

As used herein, the term “phytocannabinoid” means a cannabinoid that is naturally produced by cannabis plants, and including the acidic and decarboxylated acid forms of the naturally-occurring plant-derived cannabinoids, and also cannabinoids produced from synthetic and biosynthetic methods that are identical to naturally-occurring plant-derived cannabinoids.

The synthesis of phytocannabinoids in cannabis generally includes the following steps: (a) one or more reactions to incorporate three ketone moieties onto an acyl-CoA scaffold (in addition to the existing ketone moiety of the scaffold) (b) a reaction cyclizing the product of step (a); (c) a reaction to incorporate a prenyl moiety to the product of step (b) or a derivative of the product of step (b); and optionally (d) a reaction to cyclize the product of step (c) at the prenyl moiety. In some embodiments, the acyl moiety in the acyl-CoA scaffold comprises between four and fourteen carbons. Non-limiting examples of the acyl-CoA scaffold described in step (a) include hexanoyl-CoA and butyryl-CoA. Non-limiting examples of the product of step (b) or a derivative of the product of step (b) include olivetolic acid and divarinolic acid. In some embodiments, the prenyl moiety comprises one, two, three, or four isoprene units, preferably two or three isoprene units, even more preferably two isoprene units. In a preferred embodiment, the prenyl moiety is a geranyl moiety. Non-limiting examples of the product of step (c) include cannabigerolic acid (CBGA), and cannabigevarinolic acid (CBGVA). Non-limiting examples of the product of step (d) include tetrahydrocannabinolic acid, cannabidiolic acid, and cannabichromenic acid. In some embodiments, the product of step (c) and/or (d) may be subject to further reaction, such as esterification, hydroxylation, or glycosylation. See, for example, Angela Carvalho et al, “Designing microorganisms for heterologous biosynthesis of cannabinoids” (2017) 17:4 FEMS Yeast Research 1, Xiaozhou Luo et al “Complete biosynthesis of cannabinoids and their unnatural analogues in yeast” (2019) 567 Nature 123.

Phytocannabinoids include compounds of Formula I:

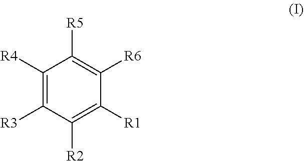

where:
R1 is a hydrogen, an optionally substituted C1-C12 alkyl, or an optionally substituted C1-C12 alkenyl;
R2 and R6 are, independently, hydrogen or carboxyl;
R3 and R5 are, independently, hydroxyl, methoxyl, ethoxyl, or halogen; and
R4 is an optionally substituted geranyl moiety;
   where R4 optionally cyclizes to R3, R5, or both.

In some embodiments, R1 is propyl or pentyl. In some embodiments, R1 is pentyl. In some embodiments, R2 is hydrogen.

Non-limiting examples of phytocannabinoids include Δ⁹-THC type, CBD type, CBG type, CBC type, CBL type, CBND type, or CBT type cannabinoids, or any combination thereof. In some embodiments, the cannabinoid material includes cannabiorcol-C1 (CBNO), CBND-C1 (CBNDO), Δ⁹-trans-Tetrahydrocannabiorcolic acid-C1 (Δ⁹-THCO), Cannabidiorcol-C1 (CBDO), Cannabiorchromene-C1 (CBCO), (−)-Δ⁸-trans-(6aR, 10aR)-Tetrahydrocannabiorcol-C1 (Δ⁸-THCO), Cannabiorcyclol C1 (CBLO), CBG-C1 (CBGO), Cannabinol-C2 (CBN-C2), CBND-C2, Δ⁹-THC-C2, CBD-C2, CBC-C2, Δ⁸-THC-C2, CBL-C2, Bisnor-cannabielsoin-C1 (CBEO), CBG-C2, Cannabivarin-C3 (CBNV), Cannabinodivarin-C3 (CBNDV), (−)-Δ⁹-trans-Tetrahydrocannabivarin-C3 (Δ⁹-THCV), (−)-Cannabidivarin-C3 (CBDV), (±)-Cannabichromevarin-C3 (CBCV), (−)-Δ⁸-trans-THC-C3 (Δ⁸-THCV), (±)-(1aS,3aR,8bR,8cR)-Cannabicyclovarin-C3 (CBLV), 2-Methyl-2-(4-methyl-2-pentenyl)-7-propyl-2H-1-benzopyran-5-ol, Δ⁷-tetrahydrocannabivarin-C3 (Δ⁷-THCV), CBE-C2, Cannabigerovarin-C3 (CBGV), Cannabitriol-C1 (CBTO), Cannabinol-C4 (CBN-C4), CBND-C4, (−)-Δ⁹-trans-Tetrahydrocannabinol-C4 (Δ⁹-THC-C4), Cannabidiol-C4 (CBD-C4), CBC-C4, (−)-trans-Δ⁸-THC-C4, CBL-C4, Cannabielsoin-C3 (CBEV), CBG-C4, CBT-C2, Cannabichromanone-C3, Cannabiglendol-C3 (OH-iso-HHCV-C3), Cannabioxepane-C5 (CBX), Dehydrocannabifuran-C5 (DCBF), Cannabinol-C5 (CBN), Cannabinodiol-C5 (CBND), (−)-Δ⁹-trans-Tetrahydrocannabinol-C5 (Δ⁹-THC), (−)-Δ⁸-trans-(6aR, 10aR)-Tetrahydrocannabinol-C5 (Δ⁸-THC), (±)-Cannabichromene-C5 (CBC), (−)-Cannabidiol-C5 (CBD), (±)-(1aS,3aR,8bR,8cR)-CannabicycloIC5 (CBL), Cannabicitran-C5 (CBR), (−)-Δ⁹-(6aS, 10aR-cis)-Tetrahydrocannabinol-C5 ((−)-cis-Δ⁹-THC), (−)-Δ⁷-trans-(1R,3R,6R)-Isotetrahydrocannabinol-C5 (trans-isoΔ⁷-THC), CBE-C4, Cannabigerol-C5 (CBG), Cannabitriol-C3 (CBTV), Cannabinol methyl ether-C5 (CBNM), CBNDM-C5, 8-OH-CBN-C5 (OH-CBN), OH-CBND-C5 (OH-CBND), 10-Oxo-Δ^{6a(10a)}-Tetrahydrocannabinol-C5 (OTHC), Cannabichromanone D-C5, Cannabicoumaronone-C5 (CBCON-C5), Cannabidiol monomethyl ether-C5 (CBDM), Δ⁹-THCM-C5, (±)-3″-hydroxy-Δ⁴″-cannabichromene-C5, (5aS,6S,9R,9aR)-Cannabielsoin-C5 (CBE), 2-geranyl-5-hydroxy-3-n-pentyl-1,4-benzoquinone-C5, 8α-Hydroxy-Δ⁹-Tetrahydrocannabinol-C5 (8α-OH-Δ⁹-THC), 8β-Hydroxy-Δ⁹-Tetrahydrocannabinol-C5 (8β-OH-Δ⁹-THC), 10α-Hydroxy-Δ⁸-Tetrahydrocannabinol-C5 (10α-OH-Δ⁸-THC), 10β-Hydroxy-Δ⁸-Tetrahydrocannabinol-C5 (10β-OH-Δ⁸-THC), 10α-hydroxy-Δ^{9,11}-hexahydrocannabinol-C5, 9β, 10β-Epoxyhexahydrocannabinol-C5, OH-CBD-C5 (OH-CBD), Cannabigerol monomethyl ether-C5 (CBGM), Cannabichromanone-C5, CBT-C4, (±)-6,7-cis-epoxycannabigerol-C5, (±)-6,7-trans-epoxycannabigerol-C5, (−)-7-hydroxycannabichromane-C5, Cannabimovone-C5, (−)-trans-Cannabitriol-C5 ((−)-trans-CBT), (+)-trans-Cannabitriol-C5 ((+)-trans-CBT), (±)-cis-Cannabitriol-C5 ((±)-cis-CBT), (−)-trans-10-Ethoxy-9-hydroxy-Δ^{6a(10a)}-tetrahydrocannabivarin-C3 [(−)-trans-CBT-OEt], (−)-(6aR,9S, 10S, 10aR)-9, 10-Dihydroxyhexahydrocannabinol-C5 [(−)-Cannabiripsol] (CBR), Cannabichromanone C-C5, (−)-6a, 7, 10a-Trihydroxy-Δ⁹-tetrahydrocannabinol-C5 [(−)-Cannabitetrol] (CBTT), Cannabichromanone B-C5, 8,9-Dihydroxy-Δ^{6a(10a)}-tetrahydrocannabinol-C5 (8,9-Di-OHCBT), (±)-4-acetoxycannabichromene-C5, 2-acetoxy-6-geranyl-3-n-pentyl-1,4-benzoquinone-C5, 11-Acetoxy-Δ 9-TetrahydrocannabinoIC5 (11-OAc-Δ 9-THC), 5-acetyl-4-hydroxycannabigerol-C5, 4-acetoxy-2-geranyl-5-hydroxy-3-npentylphenol-C5, (−)-trans-10-Ethoxy-9-hydroxy-Δ^{6a(10a)}-tetrahydrocannabinol-C5 ((−)-trans-CBTOEt), sesquicannabigerol-C5 (SesquiCBG), carmagerol-C5, 4-terpenyl cannabinolate-C5, β-fenchyl-Δ⁹-tetrahydrocannabinolate-C5, α-fenchyl-Δ⁹-tetrahydrocannabinolate-C5, epi-bornyl-Δ⁹-tetrahydrocannabinolate-C5, bornyl-Δ⁹-tetrahydrocannabinolate-C5, α-terpenyl-Δ⁹-tetrahydrocannabinolate-C5, 4-terpenyl-Δ⁹-tetrahydrocannabinolate-C5, their acidic forms. In some embodiments, the phytocannabinoids include Δ⁹-tetrahydrocannabinolic acid (“THCA”; Chemical Abstracts Service (CAS) #23978-85-0); cannabidiolic acid (“CBDA”; CAS #1244-58-1); cannabichromenic acid (“CBCA”; CAS #185505-15-1); cannabigerolic acid (“CBGA”; CAS #255555-57-1); tetrahydrocannabivarinic acid (“THCVA”; CAS #39986-26-0); cannabigerovarinic acid (“CBGVA”; CAS #64924 July 8); cannabidivarinic acid (“CBDVA”; CAS #31932-13-5); cannabichromevarinic acid (“CBCVA”; CAS #1628112-69-5); cannabinol (“CBN”, CAS #521-35-7); salts thereof; and the decarboxylated forms of the foregoing.

As used herein, the term “terpene” are molecules comprising isoprene units and, unless context dictates otherwise, includes terpenes and terpenoids. Terpenes are often volatile and provide the scent and aroma associated with essential oils of plants such as roses, citrus, cannabis, etc. Terpenes found in cannabis include: myrcene, limonene, linalool, pinene, caryophyllene, terpinolene, bisabolene, farnesene, fenchol, and guaiol. It has been postulated that the terpenes found in cannabis contribute to the “entourage effect”, where the effects of cannabinoids are modulated by the presence of the terpenes, such as by moderating the psychoactive effects of THC.

As used herein, the term “strain” means a pure or hybrid variety of cannabis, whether stabilized or not. Varieties are typically differentiated based on certain phenotypical or chemotypical traits expressed by the plant. These traits can include percentages of various cannabinoids, terpenes, powdery mildew resistance, drought tolerance, fiber content, or combinations thereof. Well-known strains of cannabis include Acapulco gold, amnesia haze, blueberry, blue dream, cannatonic, chemdawg, chrome, dance hall, Durban poison, girl scout cookies, G-13, god bud, gorilla glue, green crack, happy feet, Jack Herer, liberty haze, Nina, northern lights #5, OG Kush, pineapple express, purple kush, Raphael, skunk, Skywalker OG, sour diesel, super lemon haze, super silver haze, tangerine dream, white widow, and Willie Nelson.

As used herein, the term “strain specific” refers to a composition including a phytocannabinoid material, having a phytocannabinoid profile that is substantially similar to the phytocannabinoid profile of a particular strain of cannabis plant, a terpene material having a terpene profile that is substantially similar to the terpene profile of a strain of cannabis plant, or both. In some embodiments where a phytocannabinoid material and a terpene material are both present, the materials have a phytocannabinoid profile and a terpene profile that are substantially similar to the phytocannabinoid profile and the terpene profile of the same strain of cannabis. In some embodiments, the phytocannabinoid material and the terpene material are extracted from the same strain of cannabis, or even the same plant matter. In some embodiments, the phytocannabinoid-terpene profile is maintained as compared to phytocannabinoid-terpene profile of a cannabis plant. In other embodiments, the phytocannabinoid profile and the terpene profile are maintained as compared a cannabis plant, but not with respect to each other, e.g. there may be fewer or more terpenes present relative to the cannabinoids as compared to the cannabis plant, but the terpenes present still maintain the terpene profile of the cannabis plant.

As used herein, the term “vaporization” refers to a process by which a substance undergoes at least one phase transition to enter into a gaseous phase, as a gas, or as liquid droplets or solid particulates suspended in a gas. Unless context dictates otherwise, vaporization includes evaporation, boiling and aerosolization.

As used herein, the term “vapor” refers to a gas or a gaseous mixture including liquid droplets and/or solid particulates suspended in the gas.

### Compositions of the Present Disclosure

The present disclosure generally provides liquid compositions suitable for use in electronic vaporization devices (alternatively, “vape compositions”) comprising phytocannabinoids and terpenes.

Vape compositions are typically contained within a storage portion of the electronic vaporization device and must be transported to a vaporization section of the device where the liquid composition is vaporized, thereby allowing a user to inhale an active inhalable ingredient (“AII”) present in the vape composition. For example, a wick may draw the composition toward a heating element within such device by capillary action. The vaporization of the composition at the vaporization section creates a concentration gradient whereby the composition is urged from the storage portion toward the vaporization section. The transport of the composition along the wick is affected by the viscosity of the composition: higher viscosity compositions tend resist transport as compared to lower viscosity compositions.

Phytocannabinoid materials are often too viscous to work properly as vape compositions in conventional electronic vaporization devices, resisting the flow from the storage portion to a vaporization section. As such, conventional vape compositions with phytocannabinoid AIIs are admixed with a carrier to reduce the viscosity of the phytocannabinoid material. Conventional carriers are not endogenous to cannabis flower, and include vegetable oil, canola oil, olive oil, polyethylene glycol 400, glycerin, propylene glycol, medium chain triglycerides, triacetin, and/or triethyl citrate. Such diluents and carriers are often recognized by the US Food and Drugs Administration (USFDA) as being Generally Regarded As Safe (“GRAS”). However, GRAS status is typically determined with respect to an ingredient for administration through ingestion (e.g. when eaten), and may not have rigorous data for their use as an inhalant. As such, even where an ingredient has recognized GRAS status, it may not be desirable to inhale the ingredient (see, for example, NIOSH [2016]. Criteria for a recommended standard: occupational exposure to diacetyl and 2,3-pentanedione. By McKernan L T, Niemeier R T et al. Cincinnati, OH: U.S. Department of Health and Human Services, Centers for Disease Control and Prevention, National Institute for Occupational Safety and Health, DHHS (NIOSH) Publication No. 2016-111). Further, users have reported that the inhalation of vaporized liquid compositions that include certain diluents and carriers can create unpleasant side effects, such as sore throat or dry mouth. Accordingly, in some embodiments the liquid composition is free or substantially free (e.g. less than 5, 4, 3, 2 or 1% by weight of the vape composition) of carriers. Where the liquid composition is free or substantially free of carriers, the total material load that is inhaled into the lungs for a particular dose of AII may be lower as compared to the total material load inhaled into the lungs where the liquid composition includes carriers. Further, vaporization of vape compositions including certain carriers (such as vegetable glycerin) are more likely to result in formation of undesirable compounds, such as carbonyls, formaldehydes, acetaldehydes, etc.

Further, phytocannabinoid materials have little intrinsic flavor or aroma. As such vape compositions that consist of phytocannabinoid materials may not provide acceptable feedback to users, as they have little olfactory cues to indicate how much AII a user is intaking, no satisfaction in taking the flavor and aroma associated with the vaping experience, and are unlikely to benefit from any “entourage effect” associated with a particular strain of cannabis.

It has been found that terpenes are able to modulate the viscosity of vape compositions with phytocannabinoid AIIs with reduced (or even without) need for adscititious carriers, while simultaneously providing flavors and aromas to the vape composition.

According to an aspect of the disclosure, there is provided a liquid composition for an electronic vaporization device consisting essentially of an active inhalable source (“AIS”) comprising an active inhalable ingredient (“AII”), and a terpene source.

In some embodiments, the AIS is a cannabinoid source. In such embodiments, the AII comprises, consists essentially of, or is at least one cannabinoid. In some embodiments, the AIS is a phytocannabinoid source. In such embodiments, the AII comprises, consists essentially of, or is one or more phytocannabinoids. In some embodiments, the AII comprises, consists essentially, or is more than one phytocannabinoid.

In some of those embodiments where the AII comprises, consists essentially of, or is more than one phytocannabinoid, the AIS has a phytocannabinoid profile identical or substantially similar to that of a cannabis variety or is strain specific. By having a phytocannabinoid profile that is identical or substantially similar to a cannabis variety, the AIS may better simulate the effects of the inhalation of that cannabis variety and the entourage effects associated with that cannabis variety.

In some embodiments, the AII includes, consists essentially of, or is Δ⁹-THC type, CBD type, CBG type, CBC type, CBL type, CBND type, or CBT type cannabinoids, or any combination thereof. In some embodiments, the cannabinoid material includes, consists (CBNO), essentially of, or is cannabiorcol-C1 CBND-C1 (CBNDO), Δ⁹-trans-Tetrahydrocannabiorcolic acid-C1 (Δ⁹-THCO), Cannabidiorcol-C1 (CBDO), Cannabiorchromene-C1 (CBCO), (−)-Δ⁸-trans-(6aR,10aR)-Tetrahydrocannabiorcol-C1 (Δ⁸-THCO), Cannabiorcyclol C1 (CBLO), CBG-C1 (CBGO), Cannabinol-C2 (CBN-C2), CBND-C2, Δ⁹-THC-C2, CBD-C2, CBC-C2, Δ⁸-THC-C2, CBL-C2, Bisnor-cannabielsoin-C1 (CBEO), CBG-C2, Cannabivarin-C3 (CBNV), Cannabinodivarin-C3 (CBNDV), (−)-Δ⁹-trans-Tetrahydrocannabivarin-C3 (Δ⁹-THCV), (−)-Cannabidivarin-C3 (CBDV), (±)-Cannabichromevarin-C3 (CBCV), (−)-Δ⁸-trans-THC-C3 (Δ⁸-THCV), (±)-(1aS,3aR,8bR,8cR)-Cannabicyclovarin-C3 (CBLV), 2-Methyl-2-(4-methyl-2-pentenyl)-7-propyl-2H-1-benzopyran-5-ol, Δ⁷-tetrahydrocannabivarin-C3 (Δ⁷-THCV), CBE-C2, Cannabigerovarin-C3 (CBGV), Cannabitriol-C1 (CBTO), Cannabinol-C4 (CBN-C4), CBND-C4, (−)-Δ⁹-trans-Tetrahydrocannabinol-C4 (Δ⁹-THC-C4), Cannabidiol-C4 (CBD-C4), CBC-C4, (−)-trans-Δ⁸-THC-C4, CBL-C4, Cannabielsoin-C3 (CBEV), CBG-C4, CBT-C2, Cannabichromanone-C3, Cannabiglendol-C3 (OH-iso-HHCV-C3), Cannabioxepane-C5 (CBX), Dehydrocannabifuran-C5 (DCBF), Cannabinol-C5 (CBN), Cannabinodiol-C5 (CBND), (−)-Δ⁹-trans-Tetrahydrocannabinol-C5 (Δ⁹-THC), (−)-Δ⁸-trans-(6aR, 10aR)-Tetrahydrocannabinol-C5 (Δ⁸-THC), (±)-Cannabichromene-C5 (CBC), (−)-Cannabidiol-C5 (CBD), (±)-(1aS,3aR,8bR,8cR)-CannabicycloIC5 (CBL), Cannabicitran-C5 (CBR), (−)-Δ⁹-(6aS, 10aR-cis)-Tetrahydrocannabinol-C5 ((−)-cis-Δ⁹-THC), (−)-Δ⁷-trans-(1R,3R,6R)-Isotetrahydrocannabinol-C5 (trans-isoΔ⁷-THC), CBE-C4, Cannabigerol-C5 (CBG), Cannabitriol-C3 (CBTV), Cannabinol methyl ether-C5 (CBNM), CBNDM-C5, 8-OH-CBN-C5 (OH-CBN), OH-CBND-C5 (OH-CBND), 10-Oxo-Δ^{6a(10a)}-Tetrahydrocannabinol-C5 (OTHC), Cannabichromanone D-C5, Cannabicoumaronone-C5 (CBCON-C5), Cannabidiol monomethyl ether-C5 (CBDM), Δ⁹-THCM-C5, (±)-3″-hydroxy-Δ⁴″-cannabichromene-C5, (5aS,6S,9R,9aR)-Cannabielsoin-C5 (CBE), 2-geranyl-5-hydroxy-3-n-pentyl-1,4-benzoquinone-C5, 8α-Hydroxy-Δ⁹-Tetrahydrocannabinol-C5 (8α-OH-Δ⁹-THC), 8β-Hydroxy-Δ⁹-Tetrahydrocannabinol-C5 (8β-OH-Δ⁹-THC), 10α-Hydroxy-Δ⁸-Tetrahydrocannabinol-C5 (10α-OH-Δ⁸-THC), 10β-Hydroxy-Δ⁸-Tetrahydrocannabinol-C5 (10β-OH-Δ⁸-THC), 10α-hydroxy-Δ^{9,11}-hexahydrocannabinol-C5, 9β, 10β-Epoxyhexahydrocannabinol-C5, OH-CBD-C5 (OH-CBD), Cannabigerol monomethyl ether-C5 (CBGM), Cannabichromanone-C5, CBT-C4, (±)-6,7-cis-epoxycannabigerol-C5, (±)-6,7-trans-epoxycannabigerol-C5, (−)-7-hydroxycannabichromane-C5, Cannabimovone-C5, (−)-trans-Cannabitriol-C5 ((−)-trans-CBT), (+)-trans-Cannabitriol-C5 ((+)-trans-CBT), (±)-cis-Cannabitriol-C5 ((±)-cis-CBT), (−)-trans-10-Ethoxy-9-hydroxy-Δ^{6a(10a)}-tetrahydrocannabivarin-C3 [(−)-trans-CBT-OEt], (−)-(6aR,9S, 10S, 10aR)-9, 10-Dihydroxyhexahydrocannabinol-C5 [(−)-Cannabiripsol] (CBR), Cannabichromanone C-C5, (−)-6a, 7, 10a-Trihydroxy-Δ⁹-tetrahydrocannabinol-C5 [(−)-Cannabitetrol] (CBTT), Cannabichromanone B-C5, 8,9-Dihydroxy-Δ^{6a(10a)}-tetrahydrocannabinol-C5 (8,9-Di-OHCBT), (±)-4-acetoxycannabichromene-C5, 2-acetoxy-6-geranyl-3-n-pentyl-1,4-benzoquinone-C5, 11-Acetoxy-Δ 9-TetrahydrocannabinoIC5 (11-OAc-Δ 9-THC), 5-acetyl-4-hydroxycannabigerol-C5, 4-acetoxy-2-geranyl-5-hydroxy-3-npentylphenol-C5, (−)-trans-10-Ethoxy-9-hydroxy-Δ^{6a(10a)}-tetrahydrocannabinol-C5 ((−)-trans-CBTOEt), sesquicannabigerol-C5 (SesquiCBG), carmagerol-C5, 4-terpenyl cannabinolate-C5, β-fenchyl-Δ⁹-tetrahydrocannabinolate-C5, α-fenchyl-Δ⁹-tetrahydrocannabinolate-C5, epi-bornyl-Δ⁹-tetrahydrocannabinolate-C5, bornyl-Δ⁹-tetrahydrocannabinolate-C5, α-terpenyl-Δ⁹-tetrahydrocannabinolate-C5, 4-terpenyl-Δ⁹-tetrahydrocannabinolate-C5, their acidic forms, salts of the acidic forms, or any combination thereof.

In some embodiments, the phytocannabinoids of the AII includes, consists essentially of, or are THC, THCA, CBD, CBDA, CBG, CBGA, CBC, CBCA, THCV, THCVA, CBDV, CBDVA, CBGV, CBGVA, CBCV, CBCVA, or any combination thereof.

In some embodiments, the cannabinoid or phytocannabinoid source comprises, consists essentially of, or is a cannabis extract, at least one purified cannabis distillate, at least one purified cannabinoid isolate, at least one synthetic cannabinoid, at least one biosynthetic cannabinoid or a combination thereof. In some embodiments, the cannabinoid source is at least one cannabis extract, at least one purified cannabis isolate, at least one synthetic cannabinoid, at least one biosynthetic cannabinoid, or a combination thereof. In some embodiments, the cannabinoid source comprises, consists essentially of, or is at least one cannabis extract. In some embodiments, the cannabis extract is a winterized cannabis extract.

In some embodiments, the cannabinoid source is at least one cannabis extract that is supplemented with at least one purified cannabinoid isolate, at least one synthetic cannabinoid, at least one biosynthetic cannabinoid, or a combination thereof to achieve a consistent cannabinoid profile.

Since cannabis is an agricultural crop, the cannabinoid profile may be susceptible to variations in grow conditions such as lighting, wind, nutrients, pruning, harvest time, etc. In some embodiments, the vape composition comprises a pre-determined cannabinoid and/or terpene profile. Cannabinoid and/or terpene sources can be blended to match the predetermined cannabinoid and/or terpene profiles.

In some embodiments, the cannabinoid source is at least one cannabinoid extract, optionally admixed with a cannabinoid isolate, a synthetic cannabinoid, a biosynthetic cannabinoid, or a combination thereof, that is blended to match the predetermined cannabinoid profile. In other embodiments, at least one cannabinoid isolate, at least one synthetic cannabinoid, at least one biosynthetic cannabinoid, or a combination thereof is blended to match the predetermined cannabinoid profile.

In some embodiments, the predetermined cannabinoid profile and/or terpene profile is a strain-specific cannabinoid profile. In some embodiments, the predetermined cannabinoid profile is a profile selected to provide a particular user effect. For example, the user effect can include treatment of a number of conditions (such as seizures, inflammation, pain, PTSD, depression, migraines, anxiety, IBD, nausea, glaucoma, loss of appetite, muscle spasticity, insomnia, Lennox-Gastaut syndrome, Dravet syndrome, or any other cannabinoid treatable condition), or is associated with a particular mood (sociability, soporific, stimulating, focused, reflective, etc.).

In some embodiments, the terpene source is at least one essential oil, at least one purified terpene isolate, at least one synthetic terpene, at least one biosynthetic terpene, at least one non-cannabis botanical extract, at least one cannabis extract, or a combination thereof, that is blended to match the predetermined terpene profile. In some embodiments, the terpene source is at least one purified terpene isolate, at least one synthetic terpene, at least one biosynthetic terpene, at least one cannabis extract, or a combination thereof that is blended to match the predetermined terpene profile.

In some embodiments, the predetermined terpene profile is a strain-specific terpene profile. In some embodiments, the predetermined terpene profile is a profile selected to provide a particular user effect. For example, the user effect can include effects associated with aromatherapy.

In some embodiments, the predetermined terpene profile and the predetermined cannabinoid profile are selected to provide the same particular user effect. In other embodiments, the predetermined terpene profile and the predetermined cannabinoid profile are selected to provide different particular user effects.

At temperatures greater than about 85° C., acidic cannabinoids may undergo decarboxylation. For example, THCA begins to convert into THC at about 85° C. Such decarboxylated cannabinoids may provide effects on a user that is different and/or desirable. For example, THC may provide a user with an intoxicating feeling. However, at higher temperatures or under high vacuum (which decreases the activation energy of reactions) other reactions can also occur. Such reactions can impart unpleasant, “rubbery” or “burnt”, flavors to the cannabinoid material. Without wishing to be bound by theory, it is believed that the reaction of the cannabis phytochemicals, such as pyrolytic, oxidative, Maillard, caramelization, or other degradative reactions, contribute to such flavors. Once present, these flavors cannot be easily masked or removed from the cannabinoid material unless very high purity molecular compounds are isolated from such cannabinoid source. Accordingly, in some embodiments, cannabis-derived cannabinoid sources, other than purified cannabinoid isolates, are subject to temperatures of no greater than about 200° C., 190° C., 180° C., 170° C., 160° C., 150° C., 140° C., 130° C. or even 120° C.

Conventional distillation of cannabis typically involving heating a cannabinoid feed source to temperatures of above 200° C., typically between 220 and 260° C. and are optionally conducted under vacuum (such as at pressures of less than 10, 9, 8, 7, 6 or even 5 torr), and the vapors are condensed at temperatures of between 150° C. and 230° C. At such conditions, undesirable reactions imparting unpleasant flavors may occur. Accordingly, in some embodiments, the cannabis distillate is not used as a cannabinoid source.

At conditions conducive to imparting unpleasant flavors, the decarboxylated cannabinoids THC, CBD, and/or CBC can undergo transformations--THC can be converted into CBN or Δ⁸-tetrahydrocannabinol, CBD can be converted into CBE, and/or CBC can be converted into CBL (See, e.g. Melissa M Lewis et al, “Chemical Profiling of Medical Cannabis Extracts”, (2017) 2 ACS Omega 6091). Similarly, variants of these molecules with different chain lengths at R1 on the compound of formula (I) may undergo equivalent reactions. For example, for the C3 variants, THCV can be converted into CBV or Δ⁸-tetrahydrocannabivarin, CBDV can be converted into CBE-C₃, and/or CBCV can be converted into CBLV. This may occur for other variants, such as the C4, C2 and C1 variants. These products (and the C1-C4 variants) occur in very low amounts in the cannabis plant. While these compounds may not inherently have unpleasant flavors, elevated concentrations of these compounds may be indicative that cannabinoid source was subject to conditions conducive to the generation of unpleasant flavors (such as distillation temperatures, vacuum conditions that are too high or both). Accordingly, in some embodiments, CBN, Δ⁸-tetrahydrocannabinol, CBE, CBL, and variants thereof (e.g. variants having non-C5 chain lengths at the 4 position of aromatic ring, such as C1-4 alkyl chain length) are present in an amount of less than 50, 45, 40, 35, 30, 25, 20, 15, 10, 9, 8, 7, 6, or 5% of total cannabinoids.

In some embodiments, the cannabis extract is an alcoholic extract (i.e. extracted using an alcohol, such as methanol, ethanol, or a combination thereof), a hydrocarbon extract (i.e. extracted using a hydrocarbon such as methane, ethane, propane, or butane), a carbon dioxide extract (i.e. using carbon dioxide as the solvent, such as sub-critical or supercritical carbon dioxide), or a combination. In some embodiments, the extraction is a carbon dioxide extraction.

In some embodiments, the extract is a decarboxylated extract. Cannabis extracts for use in vape compositions are typically decarboxylated. This is because users typically consume cannabinoid vape compositions for recreational use to experience an intoxicating effect caused by THC. Cannabis contains relatively more THCA than THC, and combustion of cannabis flowers causes decarboxylation of THCA to become THC. In vape compositions, the temperatures for vaporization can cause some decarboxylation of THCA, but they may insufficient to cause appreciable conversion before it is inhaled by a user.

In some embodiments, the AIS comprises, consists essentially of, or consists of from about 45 wt % to 100 wt % cannabinoids, and from 0 wt % to about 55 wt % other phytochemicals; from about 48 wt % to about 97 wt % cannabinoids, and from about 3 wt % to about 52 wt % other phytochemicals; from about 48 wt % to about 85 wt % cannabinoids, and from about 15 wt % to about 52 wt % other phytochemicals; from about 50 wt % to about 85 wt % cannabinoids, and from about 15 to about 50 wt % other phytochemicals; from about 60 wt % to about 85 wt % cannabinoids, and from about 15 to about 40 wt % other phytochemicals; from about 70 to about 85 wt % cannabinoids and from about 15 to about 30 wt % other phytochemicals.

In some embodiments, the AIS comprises greater than about 45, 50, 55, 60, 65, 70, 75, 80, 85, 90, 95, 96, 97, 98, or 99% cannabinoids.

In some embodiments, the AIS comprises less than about 100, 99, 98, 97, 96, 95, 90, 85, or 80% cannabinoids.

In some embodiments, the AIS comprises less than 25, 20, 15, 10, 9, 8, 7, 6, 5, 4, 3, 2, or 1% non-cannabinoid cannabis phytochemicals. In a preferred embodiment, the cannabinoid source is a strain specific cannabinoid source, that has a phytocannabinoid profile identical or substantially similar to that of the strain cannabis on which it is based.

In some embodiments, the terpene source comprises an essential oil, a purified terpene isolate, a synthetic terpene, a biosynthetic terpene, a non-cannabis botanical extract, a cannabis terpene extract, or a combination thereof. In some embodiments, terpene source consists of, or consists essentially of terpene compounds naturally produced by cannabis. In some embodiments, the terpene material comprises, consists essentially of, or is a cannabis terpene extract. Terpenes can be extracted from cannabis, for example, in accordance with the methods described in U.S. Pat. No. 9,649,349 to Tucker; or Porto et al (supra). Depending on the extraction technology used, cannabis terpene extracts may include some water. In some embodiments, the cannabis terpene extract is a de-watered cannabis-terpene extract. This can be done, for example, by cooling the extract below the freezing point of water and removing the ice. In such preparations, the cannabis terpene extract may have a terpene profile similar to that of the cannabis material from which it extracted.

In some embodiments, the terpene source comprises from about 50 wt % to 100 wt % terpenes, and from 0 wt % to about 50 wt % other phytochemicals; from about 50 wt % to about 95 wt % terpenes, and from about 5 wt % to about 50 wt % other phytochemicals; or from about 70 wt % to about 95 wt % terpenes, and from about 5 wt % to about 30 wt % other phytochemicals; or from about 85 wt % to about 95 wt % terpenes, and from about 5 wt % to about 15 wt % other phytochemicals.

In some embodiments, the AIS is present in an amount of from about 85 to about 96 wt % of the liquid composition, or from about 88 to about 92 wt % of the liquid composition. In some embodiments, the terpene source is present in an amount of from about 4 to about 15 wt % of the liquid composition, or from about 8 to about 12 wt % of the liquid composition. In such amounts, the terpene source provides a desirable viscosity while providing a good aromatic profile of the composition, when inhaled post-vaporization. When the terpene source is present in an amount greater than about 15 wt % of the composition; the viscosity of the composition may be too low such that the rate of transport from the reservoir to the vaporization section is undesirably high (which could, for example, cause over saturation of a wick of a cartridge, leading to leaks); the composition, when vaporized, has an aroma that is perceived as “overbearing” and “unpleasant”; or both. Further, when the terpene material is present in an amount of less than about 4 wt %, the viscosity of the composition may be high such that the rate of transport from the reservoir to the vaporization is undesirably low; the composition, when vaporized, has an aroma of the composition is perceived as “muted”.

In some embodiments, the AIS and the terpene source are derived from the same plant. In some embodiments, the Als and the terpene source are derived from cannabis. In some embodiments, the AIS and the terpene source are derived from the same cannabis strain. In some embodiments, the AIS and the terpene source are derived from the same plant matter. In some embodiments, the AIS comprises a cannabinoid source. In some embodiments, the AIS comprises a phytocannabinoid source. In some of those embodiments where the AIS and the terpene source are both derived from cannabis, the combination of the AIS and the terpene source, when vaped, provide an “entourage effect”.

In some embodiments, the liquid composition is a strain specific composition. By providing a strain specific liquid composition, a user may be able to choose a liquid composition based on a strain that they recognize, including that strain's effect on the user when used with combustion-inhalation methods. The specific strain may have cannabinoids and terpenes present in specific ratios, which cooperate to provide an entourage effect, which they may be able to simulate with the liquid composition. For example, a user may recall that smoking “White Widow”, a strain that includes relatively high THC, low CBD, and the presence of myrcene, caryophyllene and linalool, provided the user with a calming, happy experience. A strain specific liquid composition having cannabinoid and terpene profiles identical or substantially similar to the “White Widow” cannabis strain may provide the user with a similar experience as inhalation of the combusted dried flower. Further, where the AIS and the terpene source are derived from the same plant or the same plant matter, supply of precursor materials for preparing the AIS and terpene source is simplified. Managing the supply of different precursor material requires additional complexity in inventory control, growing conditions, and/or the potential of needing to deal with multiple suppliers.

In those embodiments where the AIS, the terpene source, or both are plant extracts, non-phytocannabinoid and non-terpene phytochemicals may be present in one or both of the AIS and the terpene source. These other phytochemicals can include fats, waxes, alkaloids, flavonoids, simple and/or complex sugars, polypeptides, water, or any combination thereof. These phytochemicals may help decrease the viscosity of liquid composition as compared to when the AIS consists of AIIs, the terpene source consists of terpenes, or both. The presence of these phytochemicals may decrease the viscosity of composition such that the terpene source does not need to be included in the composition in amounts great than about 15 wt %, where the aromas and smells become “overbearing”. In contrast to adscititious carriers, these phytochemicals are endogenously produced by the plant. By being free of adscititious carriers, the liquid composition is free of added chemicals and flavors, which may be beneficial for consumer preference in promotions, or to comply with certain regulatory requirements. Additionally, certain other phytochemicals may contribute to the entourage effect of cannabis. Further still, the complexity of preparing the liquid composition is reduced, as are costs associated with purchasing potentially expensive food-grade or pharmaceutical-grade solvents.

In some embodiments, the composition includes from about 5 wt % to about 15 wt % fats and waxes, or from about 10 wt % to about 12 wt % fats and waxes. In some embodiments, the composition includes from about 5 wt % to about 10 wt % sugars and polypeptides.

In some embodiments, the AIS, the terpene source, or both are processed to remove undesirable phytochemicals. In some embodiments, the undesirable phytochemicals include excess or certain undesirable waxes, fats, sugars, polypeptides, or water.

In contrast to certain conventional compositions where a carrier oil is added to a cannabinoid distillate, in some those embodiments where the AIS includes extracts, higher molecular weight wats and fats are present in a raw extract. Higher molecular weight waxes or fats tend to have higher boiling points than the AII, and as such, may not be completely vaporized in the vaporization section of an electronic vaping device. In electronic vaping devices that rely on a wick or fluidic channels to transport the liquid composition from a reservoir to a vaporization section, these waxes and fats may accumulate and clog the wick or the channels, reducing the ability of the wick or channels to transport the liquid composition. Thus, in some embodiments, the processing includes winterization to remove such fats and waxes.

In contrast to certain conventional compositions where purified terpenes are added to a cannabinoid distillate, in some those embodiments where the terpene source includes extracts, water may be present in a raw extract. Water tends to reduce the ability of the terpene material to form a homogenous mixture with the AIM. Thus, in some embodiments, the processing includes de-watering.

In an aspect, there is provided a liquid composition for an electronic vaporization device consisting essentially of at least about 60% cannabinoids, from about 5 to about 15% terpenes, and up to about 35% non-cannabinoid, non-terpene cannabis phytochemicals.

In some embodiments, the cannabinoids are contributed by at least one cannabis extract, at least one cannabis distillate, at least one cannabinoid isolate, at least one synthetic cannabinoid, at least one biosynthetic cannabinoid, or a combination thereof. In some embodiments, the cannabinoids are contributed by at least one cannabis extract, at least one cannabis isolate, at least one synthetic cannabinoid, at least one biosynthetic cannabinoid, or a combination thereof. In some embodiments, the composition includes a predetermined cannabinoid profile, wherein the cannabis extract, the cannabis distillate, the cannabinoid isolate, the synthetic cannabinoid, the biosynthetic cannabinoid, or a combination thereof are admixed to match the predetermined cannabinoid profile. In some embodiments, the composition includes a predetermined cannabinoid profile, and the cannabis extract, the cannabinoid isolate, the synthetic cannabinoid, the biosynthetic cannabinoid, or a combination thereof are admixed to match the predetermined cannabinoid profile. In some embodiments, the predetermined cannabinoid profile is a cannabis strain-specific cannabinoid profile. In some embodiments, the predetermined cannabinoid profile is associated with a particular user effect.

In some embodiments, any cannabis extract or cannabis distillate present in the composition is processed at a temperature of no greater than 180° C., 175° C., 170° C., 165° C., 160° C., 155° C., 150° C., 145° C., 140° C., 135° C., 130° C., 125° C., or 120° C.

In some embodiments, CBL, CBN, CBE, Δ⁸-THC and non-C5 variants thereof are present at a concentration of less than 50, 45, 40, 35, 30, 25, 20, 15, 10, 9, 8, 7, 6, 5, 4, 3, 2 or 1% of total cannabinoids. In some embodiments, CBL, CBN, CBE, Δ⁸-THC and C1-C4 variants thereof are present at a concentration of less than 50, 45, 40, 35, 30, 25, 20, 15, 10, 9, 8, 7, 6, 5, 4, 3, 2 or 1% of total cannabinoids. In some embodiments, CBL, CBN, CBE, Δ⁸-THC and C3 variants thereof are present at a concentration of less than 50, 45, 40, 35, 30, 25, 20, 15, 10, 9, 8, 7, 6, 5, 4, 3, 2 or 1% of total cannabinoids. In some embodiments, CBL, CBN, CBE, and Δ⁸-THC are present at a concentration of less than 50, 45, 40, 35, 30, 25, 20, 15, 10, 9, 8, 7, 6, 5, 4, 3, 2 or 1% of total cannabinoids.

In some embodiments, the terpenes are contributed by at least one essential oil, at least one purified terpene isolate, at least one synthetic terpene, at least one biosynthetic terpene, at least one non-cannabis botanical extract, at least one cannabis extract, or a combination thereof. In some embodiments, the terpenes are contributed by at least one purified terpene isolate, at least one synthetic terpene, at least one biosynthetic terpene, at least one cannabis extract, or a combination thereof.

In some embodiments, composition includes a predetermined terpene profile, and the essential oil, the purified terpene isolate, the synthetic terpene, the biosynthetic terpene, the non-cannabis botanical extract, the cannabis extract, or combination thereof are admixed to match the predetermined terpene profile. In some embodiments, the composition includes a predetermined terpene profile, wherein the purified terpene isolate, synthetic terpene, biosynthetic terpene, cannabis extract, or combination thereof are admixed to match the predetermined terpene profile. In some embodiments, the predetermined terpene profile is a cannabis strain-specific terpene profile. In some embodiments, the predetermined terpene profile is associated with a particular user effect.

In some embodiments, the composition comprises at least 300, 350, 400, 450, 500, 550, 600, or 650 mg/ml of total cannabinoids.

In some embodiments, the cannabinoids are present in an amount of greater than about 65, 70, 75, 80 or 85% by total weight of the composition. In some embodiments, the cannabinoids are present in an amount of from about 65% to about 85% by total weight of the composition. In some embodiments, the cannabinoids are present in an amount of from about 65% to about 80% by total weight of the composition. In some embodiments, the cannabinoids are present in an amount of from about 65% to about 75% by total weight of the composition.

In some embodiments, the non-cannabinoid, non-terpene cannabis phytochemicals are present in an amount of from about 15% to about 30% by total weight of the composition. In some embodiments, the non-cannabinoid, non-terpene cannabis phytochemicals are present in an amount of from about 20% to about 25% by total weight of the composition.

In some embodiments, the terpenes are present in an amount of from about 8 to about 12% by total weight of the composition.

In some embodiments, the vape composition comprises at least 300 mg/ml, or at least 350 mg/ml, or at least 400 mg/ml, or at least 450 mg/ml, or at least 500 mg/ml, or at least 550 mg/ml, or at least 600 mg/ml, or at least 650 mg/ml, of total cannabinoids.

In some embodiments, the terpenes present in the vape composition are those that occur naturally in cannabis. In some embodiments, the terpene source includes myrcene, limonene, linalool, pinene, caryophyllene, terpinolene, bisabolene, farnesene, fenchol, guaiol or any combination thereof.

Since the vape compositions are vaporized for inhalation, there exists a risk that if the flash point of the vape composition is lower than the vaporization point, an ignition source can ignite the vapors, causing injuries to the user. Accordingly, in some embodiments, the vape composition has a lower vaporization temperature than flash point.

In an aspect, there is provided a method to prepare a liquid composition for an electronic vaporization device. An AIS is brought to a temperature of from about 40° C. to about 80° C. A terpene source is admixed with the AIS.

At SATP, the viscosity of the AIS is too high, reducing the efficiency of the admixing. If the AIS is brought to a temperature of from about 40° C. to about 80° C., the viscosity of the AIS is lowered thereby reducing admixing times. However, at temperatures of greater than about 80° C., evaporation of terpenes in the terpene material, when admixed with the AIS, increases such that there is undesirable loss of terpenes.

In some embodiments, the admixing comprises stirring, high shear mixing, pressure homogenization, sonication, or a mixture thereof.

In some embodiments, the AIS to terpene source is added in a weight ratio of from about 85:8 to about 96:4, preferably from about 88:12 to about 92:8.

In some embodiments, the liquid composition is strain specific for cannabinoid source, terpene source, or both.

In an aspect, there is provided an electronic vaping device comprising the liquid composition as described above.

In an aspect, there is provided a cartridge for an electronic vaping device comprising the liquid composition as described above.

In an aspect, there is provided a process of obtaining a composition from feedstocks. The composition comprises at least 80 weight % cannabinoid source and at least 5 weight % terpene source. The cannabinoid source consists of or consists essentially at least one cannabinoid. The terpene source consists of or consists essentially of at least one terpene. The converting is effected at a temperature of less than 160° C.

In some embodiments, the converting includes admixing the cannabinoid material and the terpene material. In some embodiments, the admixing comprises heating the cannabinoid material to a temperature of from about 40 to about 80° C. In some embodiments, the admixing comprises sonication.

In some embodiments, the converting includes decarboxylation of the cannabinoid source.

In some embodiments, the converting includes extraction of cannabinoids from cannabis plant matter.

### EXAMPLES

### Example 1—Preparation of Liquid Composition

Sample compositions were prepared according to the following amounts set out in Table 1, below.

TABLE 1
Sample Compositions

| Component | SC 1 | SC 2 | SC 3 | SC 4 | SC 5 | SC 6 | SC 7 | SC 8 | SC 9 | SC 10 |
|---|---|---|---|---|---|---|---|---|---|---|
| Cannabinoid | 99.5 | 98 | 96 | 94 | 92 | 90 | 88 | 86 | 84 | 82 |
| Source (g) | | | | | | | | | | |
| Terpene | 0.5 | 2 | 4 | 6 | 8 | 10 | 12 | 14 | 16 | 18 |
| Source (g) | | | | | | | | | | |

The sample compositions were prepared by heating the cannabinoid source to a temperature of 60° C. The terpene material was then admixed with the heated cannabinoid material using sonication for 5 minutes and then allowed to cool.

The cannabinoid source is a decarboxylated cannabis cannabinoid extract obtained from a White Widow cannabis variety using supercritical CO₂ extraction, with terpenes first extracted from the plant matter using CO₂ extraction. The cannabinoid source has the cannabinoid profile as set out in Table 2, below. The cannabinoid source consisted of about 80% phytocannabinoids and about 20 wt % non-cannabinoid phytochemicals.

TABLE 2
Relative Amounts of Phytocannabinoids
in White Widow Cannabinoid Source

| Cannabinoid | Relative % |
|---|---|
| CBG | 5% |
| CBN | 3% |
| THC | 88% |
| CBC | 4% |

The terpene source is the cannabis terpene extract described above, that has been de-watered. The cannabis terpene extract has the terpene profile as set out in Table 3, below. Although a number of terpenes are quantified, there are additional terpenes that may be present. Terpenes, even in minute amounts (e.g. on the order of ppm), can contribute to the overall smell and aroma of a composition. The terpene source includes about 10 wt % non-terpene phytochemicals.

TABLE 3
Relative Amounts of Terpenes in White Widow Terpene Source

| Terpene | Relative % |
|---|---|
| Alpha-Pinene | 1% |
| Beta-Pinene | 1% |
| Beta-Myrcene | 31% |
| Limonene | 5% |
| Beta-Ocimene | 3% |
| Fenchone Isomers | 2% |
| Linalool | 5% |
| Fenchyl Alcohol | 1% |
| Borneol Isomers | 2% |
| Alpha-Terpineol | 2% |
| Trans-Caryophyllene | 30% |
| Alpha-Humulene | 9% |
| Guaiol | 3% |
| Alpha-Bisabolol | 4% |

### Example 2A—User Perception Testing of Proxy Compositions

Proxy compositions similar to Sample Compositions 1-10 as described in Examples 1 were prepared (Sample Compositions 1A-10A), but substituting the White Widow cannabis source with a hemp extract with the same phytocannabinoid: non-phytocannabinoid ratio obtained from Mile High Labs, and substituting the White Widow terpene source with a purified terpene isolate. Samples compositions 1A-10A were loaded into a CCELL™ TH2 cartridge matched with a CCELL™ M3 battery. A panel of participants were asked to provide feedback on the strength of the flavors. Comments from the participants were aggregated and set out in Table 4, below.

TABLE 4
Aggregated participant comments

| SC 1A | SC 2A | SC 3A | SC 4A | SC 5A |
|---|---|---|---|---|
| very light | light | nice flavor | very nice | very nice |
| flavor | flavor- | | flavor | flavor |
| SC 6A | SC 7A | SC8A | SC9A | SC10A |
| very nice | nice | very strong | very strong | overbearing |
| flavor | flavor | flavor | flavor | |

### Example 2B—User Perception Testing Against Commercially Available Products

A panel of participants smelled the terpene source of the Sample Compositions described in Example 1 and compared them with commercially available strain specific vape compositions available from Jetty, Island and Bloom Farms that were purchased from MedMen™ retail stores in California.

The participants noted that as compared to the commercially available compositions:
Terpene source of Example 1 better replicated the flavors of the strains on which the composition was based, noting that some of the commercially available compositions had a “chemical” or “artificial” flavor profile; and.
The Terpene source of Example 1 better replicated the smells and aroma of the dried flower of the strain from which the liquid composition is derived.

### Example 3

Having reference to FIG. 2, a pump 200 was set to draw a series “puffs” from a vaping device 210 (using a M3B battery commercially available from CCELL™) to simulate use by a vape user. The mouthpiece of the vaping device 210 was fluidically connected to an inline filter 220 (Whatman™ grade f319-04 filter paper) to collect particulate matter generated by the vaping device 210. An impinger 230 containing a liquid material 240 (impinger liquid) was fluidically connected downstream of the filter 220 to collect aerosol components not trapped by the filter 220.

### Experiment Parameters:

In the experiments, the pump 200 was configured to draw a series puffs, each having a volume of 120 mL and a duration of 5 s, and at a puff interval of 60 s. Groups of 10 puffs were aggregated into segments. After each segment, the pump 200 was paused to allow the contents of filter 220 and the fluid material 940 to be removed. The filter 220 was measured before and after each segment, and the increase in mass is defined as the “aerosol mass”. The filter 220 and the liquid material 240 was replaced with a fresh filter and liquid. The pump 200 continued to draw puffs in segments until the aerosol mass in a segment was less than 0.5 mg/puff.

The experiment was re-run for each analyte of interest (cannabinoids and carbonyls). The liquid material 240 was varied for each analyte.

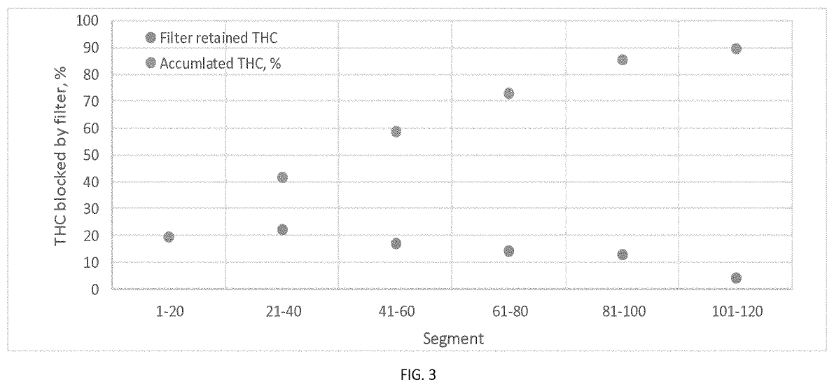

To test for cannabinoids, the cartridge of the device 210 was a M6T05 cartridge from CCELL™, and the liquid material 240 was initially 15 mL MeOH. The filter 220 was rinsed with 20 mL of MeOH and the liquid material 240 was collected. The filter and liquid material were replaced between each segment. HPLC was used to determine the cannabinoid content in the liquid material 240 and the eluate of the filter 220, and analyzed separately. The percent of cannabinoids collected in each segment, relative to the cannabinoids present in the vape composition, is set out in FIG. 3.

To test for carbonyls, the cartridge of the device 210 was a TH2 cartridge from CCELL™, and the liquid material 240 was initially 10 mL H₂O (the “collection water”). The filter 220 was immersed into the collection water to dissolve any carbonyls captured in the filter 220 into the collection water. The filter and collection water were replaced between each segment. The total carbonyl content in the collection water, comprising both the carbonyls trapped by the filter and the carbonyls collected in the liquid material 240, was tested by GCMS.

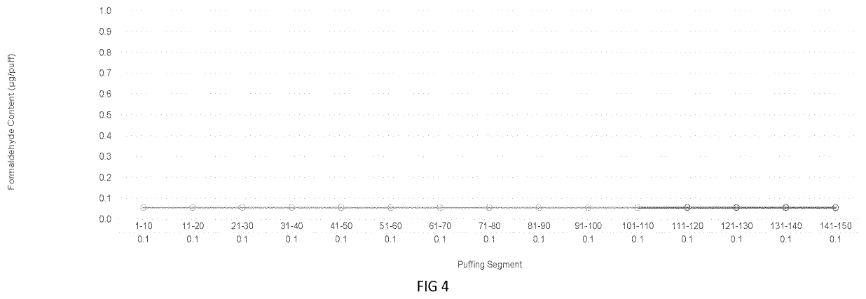

As shown in FIG. 4, an average of less than 0.1 μg of formaldehyde was detected per puff. In contrast, approximately 8.5 μg of formaldehyde are generated per puff of cigarettes, and electronic cigarettes typically generate under 1 μg of formaldehyde per puff, but up to about 15 μg of formaldehyde per puff.

Every document referenced herein, including publications and published patent documents, is hereby incorporated by reference herein in its entirety unless expressly excluded or otherwise limited. Reference to any document is not an admission that it is prior art with respect to any invention disclosed or claimed herein or that it alone, or in any combination with any other reference or references, teaches, suggests or discloses any such invention. Further, to the extent that any meaning or definition of a term in this document conflicts with any meaning or definition of the same term in a document incorporated by reference, the meaning or definition assigned to that term in this document will govern.

It is to be understood that the present disclosure, including description and drawings, are provided for the purpose of illustration and as an aid to understanding. The scope of the invention should not be limited in scope any embodiments or examples provided in the present disclosure, such as the application, details of construction or arrangements of the components. Except to the extent explicitly stated or inherent within the processes described, including any optional steps or components thereof, no required order, sequence, or combination is intended or implied. The invention is capable of other embodiments and of being practiced and carried out in various ways. Also, it is to be understood that the phraseology and terminology employed herein are for the purpose of description and should not be regarded as limiting. The scope of the claims should not be limited by the preferred embodiments set forth in the disclosure but should be given the broadest interpretation consistent with the disclosure as a whole.

1-58. (canceled)

59. A method of manufacturing an electronic vaporizer or electronic vaporizer cartridge comprising filling the electronic vaporizer or electronic vaporizer cartridge with a composition consisting essentially of:
a) greater than about 60 wt % cannabinoids;
b) from about 5 wt % to about 15 wt % terpenes; and
c) less than about 35 wt % non-cannabinoid, non-terpene cannabis phytochemicals, based on the total weight of the liquid composition.

60. The method of claim 59, wherein the cannabinoids have a cannabis.

61. The method of claim 59, wherein Cannabinol-C5 (CBN), D8-tetrahydrocannabinol, (5aS,6S,9R,9aR)-Cannabielsoin-C5 (CBE), (±)-(1aS,3aR,8bR,8cR)-CannabicyclolC5 (CBL), and non-C5 chain length variants thereof are present in an amount of less than 50% of the total cannabinoids.

62. The method of claim 59, wherein the terpenes have a cannabis strain-specific terpene profile.

63. The method of claim 59, wherein the non-cannabinoid, non-terpene cannabis phytochemicals are present in an amount of from about 15% to about 30% by total weight of the composition.

64. The method of claim 59, wherein the cannabinoids are present in an amount of from about 65% to about 85% by total weight of the composition.

65. The method of claim 59, wherein the terpenes are present in an amount of from about 8% to about 12% by total weight of the composition.

66. The method of claim 59, wherein the composition comprises:
a) an active inhalable source (AIS) comprising the cannabinoids; and
b) a terpene source comprising the terpenes,
wherein the non-cannabinoid, non-terpene cannabis phytochemicals are present in the AIS, the terpene source, or both.

67. The method of claim 66, wherein the ratio between the AIS and the terpene source is from about 85:15 to about 96:4.

68. The method of claim 66, wherein the AIS consists essentially of:
a) greater than 70% cannabinoids by total weight of the AIS, and
b) less than 30 wt % other cannabis phytochemicals by total weight of the AIS.

69. The method of claim 66, wherein the terpene source consists essentially of:
a) from about 85 wt % to about 95 wt % terpenes, and
b) from about 5 wt % to about 15 wt % other phytochemicals.

70. The method of claim 66, wherein the active inhalable source is at least one cannabis extract, at least one purified cannabis distillate, at least one purified cannabinoid isolate, at least one synthetic cannabinoid, at least one biosynthetic cannabinoid, or a combination thereof.

71. The method of claim 70, wherein the active inhalable source comprises at least one cannabis extract.

72. The method of claim 66, wherein at least one of the AIS, the terpene source and the liquid composition is strain specific.

73. The method of claim 59, wherein the liquid composition is free of adscititious carriers.

74. The method of claim 66, wherein the terpene source is at least one terpene extract, at least one purified terpene, at least one a synthetic terpene or a mixture thereof.

75. The method of claim 74, wherein the terpene extract comprises a cannabis terpene extract.

76. The method of claim 59, wherein the terpenes comprise are selected from myrcene, limonene, linalool, pinene, caryophyllene, terpinolene, bisabolene, farnesene, fenchol, guaiol or any combination thereof.

77. The method of claim 76, wherein the adscititious carriers are selected from any one of vegetable oil, canola oil, olive oil, polyethylene glycol 400, glycerin, propylene glycol, medium chain triglycerides, triacetin, and/or triethyl citrate.

78. A cartridge for an electronic vaping device or an electronic vaping device made according to the method of claim 59.
